(19) United States
(12) Patent Application Publication
(10) Pub. No.: US 2004/0147188 A1
(43) Pub. Date: Jul. 29, 2004

(54) Fluorochemical urethane composition for treatment of fibrous substrates

(75) Inventors: Mitchell T. Johnson, Hudson, WI (US); Larry A. Lien, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/352,613

(22) Filed: Jan. 28, 2003

(51) Int. Cl. C08G 77/22; B32B 5/02; B32B 27/04; B32B 27/12; B32B 1/00; D06N 7/04; B32B 27/00; B32B 27/40; B32B 9/04; C07C 213/00; C07C 215/00; C07C 217/00

(52) U.S. Cl. 442/93; 564/505; 528/30; 428/421; 428/149; 428/423.1; 428/423.5; 428/423.7; 428/447; 442/79; 442/81; 442/82; 442/94; 442/87; 442/88

(57) ABSTRACT

The compositions comprise one or more fluorochemical urethane compounds and one or more silsesquioxane compounds. This fluorochemical urethane compound(s) comprises the reaction product of (a) one or more polyfunctional isocyanate compounds; (b) one or more fluorochemical monofunctional compounds; and optionally (c) one or more hydrophilic polyoxyalkylene compounds; and/or (d) one or more silane compounds.

### FIELD OF THE INVENTION

This invention relates to fluorochemical compositions comprising one or more fluorochemical urethane compounds, and one or more silsesquioxane compounds for treatment of a fibrous substrate. The fluorochemical compositions are capable of improving one or more of the oil- and/or water repellency, stain- and/or soil repellency and stain and/or soil release properties, with improved durability, of a fibrous substrate treated with the fluorochemical composition.

### BACKGROUND OF THE INVENTION

The use of certain fluorochemical compositions on fibers and fibrous substrates, such as textiles, paper, and leather, to impart oil- and water-repellency and soil- and stain-resistance is well known in the art. See, for example, Banks, Ed., Organofluorine Chemicals and Their Industrial Applications, Ellis Horwood Ltd., Chichester, England, 1979, pp. 226-234. Such fluorochemical compositions include, for example, fluorochemical guanidines (U.S. Pat. No. 4,540,497, Chang et al.), compositions of cationic and non-cationic fluorochemicals (U.S. Pat. No. 4,566,981, Howells), compositions containing fluorochemical carboxylic acid and epoxidic cationic resin (U.S. Pat. No. 4,426,466, Schwartz), fluoroaliphatic carbodiimides (U.S. Pat. No. 4,215,205, Landucci), fluoroaliphatic alcohols (U.S. Pat. No. 4,468,527, Patel), fluorine-containing addition polymers, copolymers, and macromers (U.S. Pat. Nos. 2,803,615; 3,068,187; 3,102,103; 3,341,497; 3,574,791; 3,916,053; 4,529,658; 5,216,097; 5,276,175; 5,725,789; 6,037,429), fluorine-containing phosphate esters (U.S. Pat. Nos. 3,094,547; 5,414,102; 5,424,474), fluorine-containing urethanes (U.S. Pat. Nos. 3,987,182; 3,987,227; 4,504,401; 4,958,039), fluorochemical allophanates (U.S. Pat. No. 4,606,737) fluorochemical biurets (U.S. Pat. No. 4,668,406), fluorochemical oxazolidinones (U.S. Pat. No. 5,025,052), and fluorochemical piperazines (U.S. Pat. No. 5,451,622).

U.S. Pat. No. 3,493,424 (Mohrlok et al.) describe fibrous materials which are given antislip, dulling, and/or dry-soiling resistance properties by applying a colloidal suspension of a silsesquioxane, followed by drying the material. U.S. Pat. No. 4,351,736 (Steinberger et al.) describe a textile pile-stabilizing impregnating agent comprising a colloidal suspension of silicic acid and organosilsesquioxanes. U.S. Pat. No. 4,781,844 (Kortmann et al.) describe a textile finishing agent comprising an aqueous colloidal suspension of an organosilsesquioxane-containing sol and an organic polymer resin containing perfluoroalkyl groups which imparts soil resistance.

Solvent and water based fluorochemical compositions have been used to provide water- and oil-repellency to fibrous surfaces. Since organic solvents pose health, safety, and environmental concerns, the water-based compositions are particularly desirable. However, the previously known compositions are typically aqueous dispersions or emulsions, not solutions; therefore, may require a high temperature cure to impart good repellency properties. In many cases, for example, high temperature curing is not practical or possible. For this reason there is a continuing need for fluorochemical urethanes that do not require costly and energy consuming high temperature cure conditions to impart good repellency properties.

### SUMMARY OF THE INVENTION

In one aspect, this invention relates to fluorochemical compositions comprising one or more fluorochemical urethane compounds, and one or more silsequioxane compounds, wherein said silsesquioxane comprises less than 10 wt. % of cocondensates of tetralkoxysilanes (or hydrosylates thereof, e.g. Si(OH)₄). Preferably, the silsesquioxane comprises less than 5 wt. %, more prepferably less than 2 wt. % of of cocondensates of tetralkoxysilanes or hydrosylates thereof. Significantly, the composition provides superior water-repellency without sacrificing oil repellency. Although polyorganosilanes, such as poly(dimethylsiloxane), have been used to impart water-repellency to and improve the “hand” or softness of fibrous substrates, they are known to have a deleterious effect on oil- and or soil-repellency.

These fluorochemical urethane compounds comprise the reaction product of (a) one or more polyfunctional isocyanate compounds; and (b) one or more fluorochemical monofunctional compounds. Optionally, the fluorochemical urethane compounds further comprise (c) one or more hydrophilic polyoxyalkylene compounds; and/or (d) one or more isocyanate-reactive silanes.

The inventors recognized the need for fluorochemical compositions that can successfully impart one or more of the following uniform, durable properties: oil- and water-repellency and/or soil- and stain-resistance and/or soil- and stain-repellency. These chemical compositions may be water and/or organic solvent soluble or dispersible and, in many embodiments, may be cured under ambient temperatures.

Another embodiment of the present invention relates to a composition for treatment of fibrous substrates comprising a solution or dispersion of the fluorochemical composition of the present invention and a solvent. Preferably the fluorochemical composition is soluble in the solvent. When applied to a substrate, this treatment composition provides a uniform distribution of the chemical composition on the substrate without altering the appearance of the substrate. With some embodiments a high temperature cure is not required to provide this coating; the treatment composition can be cured (i.e. dried) at ambient temperatures. In other embodiments a high temperature cure (e.g. temperatures at or above about 125° F. or 49° C.) may be used with coating compositions of the invention.

These compositions can be applied as coatings to a wide variety of substrates, for example, by topical application, to impart durable release/repellency/resistant properties to the substrates. When applied as a coating, the chemical compositions of the present invention can provide uniform properties to a fibrous substrate and do not change the appearance of the substrate to which they are applied. Even though the urethane compounds are of relatively low fluorochemical content, the chemical compositions of the present invention provide durable stain-release properties comparable to or better than those of the prior art. In addition, with some embodiments, the chemical compositions of the present invention do not require high temperature curing; they can be cured (i.e., dried) at ambient temperature.

This invention also relates to an article comprising a fibrous substrate having a cured coating derived from at least one solvent and a chemical composition of the present invention. After application and curing of the chemical composition, the substrate displays durable release/resistance/repellency properties.

This invention further relates to a method for imparting stain-release and/or repellency characteristics to a fibrous substrate, having one or more surfaces, by applying the coating composition of the present invention onto one or more surfaces of the substrate and allowing the coating composition to cure (i.e. dry).

Unless otherwise stated, the following terms used in the specification and claims have the meanings given below:

“Acyloxy” means a radical —OC(O)R where R is, alkyl, alkenyl, and cycloalkyl, e.g., acetoxy, 3,3,3-trifluoroacetoxy, propionyloxy, and the like.

“Alkoxy” means a radical —OR where R is an alkyl group as defined below, e.g., methoxy, ethoxy, propoxy, butoxy, and the like.

“Alkyl” means a linear saturated monovalent hydrocarbon radical having from one to about twelve carbon atoms or a branched saturated monovalent hydrocarbon radical having from three to about twelve carbon atoms, e.g., methyl, ethyl, 1-propyl, 2-propyl, pentyl, and the like.

“Alkylene” means a linear saturated divalent hydrocarbon radical having from one to about twelve carbon atoms or a branched saturated divalent hydrocarbon radical having from three to about twelve carbon atoms, e.g., methylene, ethylene, propylene, 2-methylpropylene, pentylene, hexylene, and the like.

“Aryl aliphatic” means an alkylene radical defined above with an aromatic group attached to the alkylene radical, e.g., benzyl, pyridylmethyl, 1-naphthylethyl, and the like.

“Cured chemical composition” means that the chemical composition is dried or solvent has evaporated from the chemical composition at ambient temperature (15-35° C.) for up to approximately 24 hours or at elevated temperature until dryness.

“Fibrous substrate” means materials comprised of synthetic fibers such as wovens, knits, nonwovens, carpets, and other textiles; and materials comprised of natural fibers such as cotton, paper, and leather.

“Fluorocarbon monofunctional compound” means a compound having one isocyanate-reactive functional group and a perfluoroalkyl or a perfluoroheteroalkyl group (including perfluoropolyethers), e.g. C₄F₉SO₂N(CH₃)CH₂CH₂OH, C₄F₉SO₂N(CH₃)CH₂CH₂NH₂, C₄F₉CH₂CH₂OH, C₄F₉CH₂CH₂SH, C₂F₅O(C₂F₄O)₃CF₂CONHC₂H₄OH, C₂F₅O(C₂F₄O)₃CF₂CONHC₂H₄CO₂H, C₆F₁₃CH₂OH, C₆F₁₃CH₂N(CH₃)OH, and the like.

“Fluorochemical urethane compound” means a compound derived or derivable from the reaction of at least one polyfunctional isocyanate compound and one or more fluorinated monofunctional compounds; and optionally at least one hydrophilic polyoxyalkylene compound, and/or one or more isocyanate-reactive silane compounds.

“Heteroacyloxy” has essentially the meaning given above for acyloxy except that one or more heteroatoms (i.e. oxygen, sulfur, and/or nitrogen) may be present in the R group and the total number of carbon atoms present may be up to 50, e.g., CH₃CH₂OCH₂CH₂C(O)O—, C₄H₉OCH₂CH₂OCH₂CH₂C(O)O—, CH₃O(CH₂CH₂O)ₙCH₂CH₂C(O)O—, and the like.

“Heteroalkoxy” has essentially the meaning given above for alkoxy except that one or more heteroatoms (i.e. oxygen, sulfur, and/or nitrogen) may be present in the alkyl chain and the total number of carbon atoms present may be up to 50, e.g. CH₃CH₂OCH₂CH₂O—, C₄H₉OCH₂CH₂OCH₂CH₂O—, CH₃O(CH₂CH₂O)ₙH, and the like.

“Heteroalkyl” has essentially the meaning given above for alkyl except that one or more catenary heteroatoms (i.e. oxygen, sulfur, and/or nitrogen) may be present in the alkyl chain, these heteroatoms being separated from each other by at least one carbon, e.g., CH₃CH₂OCH₂CH₂—, CH₃CH₂OCH₂CH₂OCH(CH₃)CH₂—, C₄F₉CH₂CH₂SCH₂CH₂—, and the like.

“Heteroalkylene” has essentially the meaning given above for alkylene except that one or more catenary heteroatoms (i.e. oxygen, sulfur, and/or nitrogen) may be present in the alkylene chain, these heteroatoms being separated from each other by at least one carbon, e.g., —CH₂OCH₂O—, —CH₂CH₂OCH₂CH₂—, —CH₂CH₂N(CH₃)CH₂CH₂—, —CH₂CH₂SCH₂CH₂—, and the like.

“Heteroaralkylene” means an aralkylene radical defined above except that catenary oxygen, sulfur, and/or nitrogen atoms may be present, e.g., phenyleneoxymethyl, phenyleneoxyethyl, benzyleneoxymethyl, and the like.

“Halo” means fluoro, chloro, bromo, or iodo, preferably fluoro and chloro.

“Isocyanate-reactive functional group” means a functional group that is capable of reacting with an isocyanate group, such as hydroxyl, amino, thiol, etc.

“Perfluoroalkyl” has essentially the meaning given above for “alkyl” except that all or essentially all of the hydrogen atoms of the alkyl radical are replaced by fluorine atoms and the number of carbon atoms is from 2 to about 12, e.g. perfluoropropyl, perfluorobutyl, perfluorooctyl, and the like.

“Perfluoroalkylene” has essentially the meaning given above for “alkylene” except that all or essentially all of the hydrogen atoms of the alkylene radical are replaced by fluorine atoms, e.g., perfluoropropylene, perfluorobutylene, perfluorooctylene, and the like

“Perfluoroheteroalkyl” has essentially the meaning given above for “heteroalkyl” except that all or essentially all of the hydrogen atoms of the heteroalkyl radical are replaced by fluorine atoms and the number of carbon atoms is from 3 to about 100, e.g. CF₃CF₂OCF₂CF₂—, CF₃CF₂O(CF₂CF₂O)₃CF₂CF₂—, C₃F₇O(CF(CF₃)CF₂O)ₘCF(CF₃)CF₂— where m is from about 10 to about 30, and the like.

“Perfluoroheteroalkylene” has essentially the meaning given above for “heteroalkylene” except that all or essentially all of the hydrogen atoms of the heteroalkylene radical are replaced by fluorine atoms, and the number of carbon atoms is from 3 to about 100, e.g., —CF₂OCF₂—, —CF₂O(CF₂O)ₙ(CF₂CF₂O)ₘCF₂—, and the like.

“Perfluorinated group” means an organic group wherein all or essentially all of the carbon bonded hydrogen atoms are replaced with fluorine atoms, e.g. perfluoroalkyl, perfluoroheteroalkyl, and the like.

“Polyisocyanate compound” means a compound containing two or more isocyanate radicals, —NCO, attached to a multivalent organic group, e.g. hexamethylene diisocyanate, the biuret and iscyanurate of hexamethylene diisocyanate, and the like.

“Reactive polyoxyalkylene” means a polymer having oxyalkylene repeat units with an average of 1 or more isocyanate-reactive functional groups per molecule.

“Silane group” means a group comprising silicon to which at least one hydrolyzable group is bonded, e.g. —Si(OCH₃)₃, —Si(OOCCH₃)₂CH₃, —Si(Cl)₃, and the like.

“Repellency” is a measure of a treated substrate's resistance to wetting by oil and/or water and or adhesion of particulate soil. Repellency may be measured by the test methods described herein.

“Resistance” is a measure of the treated substrate's ability to avoid staining and/or soiling when contacted by stain or soil respectively.

“Release” is a measure of the treated substrate's ability to have soil and/or stain removed by cleaning or laundering.

“Release/resistance/repellency” means the composition demonstrates at least one of oil repellency, water repellency, stain release, stain repellency, soil release and soil repellency.

“Silsesquioxane” and “silsesquioxane cocondensates” are cocondensates of dialkoxysilanes (or hydrosylates thereof) of the formula R¹⁰₂Si(OR¹¹)₂ with trialkoxysilanes (or hydrosylates thereof) of the formula R¹⁰SiO_{3/2} where each R¹⁰ is an alkyl group of 1 to 6 carbon atoms or an aryl group and R¹¹ represents an alkyl radical with 1 to 4 carbon atoms. The silsesquioxane may optional further comprise a co-condensate of silanes of the formula R¹⁰₃SiOR¹¹.

### DETAILED DESCRIPTION OF THE INVENTION

The chemical compositions of the present invention comprise one or more fluorochemical urethane compounds and one or more silsesquioxane compounds, for improving the resistance/release/repellency of a fibrous substrate treated with the fluorochemical urethane compounds. This fluorochemical urethane compound comprises the reaction product of (a) one or more polyfunctional isocyanate compounds; (b) one or more fluorochemical monofunctional compounds; and optionally (c) one or more hydrophilic polyoxyalkylene compounds; and/or (d) one or more silane compounds.

Generally, the weight ratio of said first component to said second component is from 1:1 to 110:1, preferably 3:1 to 5:1. More particularly, the composition may comprise 50 to 90 wt. % of said first component and 15 to 50 wt. % of said second component.

Each fluorochemical urethane compound comprises a urethane group that is derived or derivable from the reaction of at least one polyfunctional isocyanate compound and at least one fluorochemical monofunctional compound. The fluorochemical urethane compound is terminated, on average, with (a) one or more perfluoroalkyl groups, (b) one or more perfluoroheteroalkyl groups; and optionally (c) one or more silyl groups and/or (d) one or more hydrophilic polyoxyalkylene groups. It will be understood that the reaction product will provide a mixture of compounds, some percentage of which will comprise compounds as described, but may further comprise urethane compounds having different substitution patterns and degree of substitution.

Generally, the amount of said fluorochemical monofunctional compounds is sufficient to react with between 60 and 95% of available isocyanate groups, and if present, the amount of said hydrophilic polyoxyalkylene compound is sufficient to react with between 0.1 and 30% of available isocyanate groups, and the amount of said silanes is sufficient to react with between 0.1 and 25% of available isocyanate groups. Preferably, the amount of said hydrophilic polyoxyalkylene(s) is sufficient to react with between 0.5 and 30% of available isocyanate groups, the amount of said silanes is sufficient to react with between 0.1 and 15% of available isocyanate groups, and the amount of said fluorochemical monofunctional compounds is sufficient to react with between 60 and 90% of available isocyanate groups.

Preferred classes of urethane compounds that may be present are represented by the following formulas:

R_{f}ZR²—X′(—CONH-Q(A)ₘ-NHCO—X′R³X′—)ₙCONH-Q(A)-NHCO—X′R¹Si(Y)₃

R_{f}ZR²—X′(—CONH-Q(A)ₘ-NHCO—X′R³X′—)ₙCONHR¹Si(Y)₃

wherein:

R_{f}ZR² is a residue of at least one of the fluorochemical monofunctional compounds;

R_{f} is a perfluoroalkyl group having 2 to about 12 carbon atoms, or a perfluoroheteroalkyl group having 3 to about 50 carbon atoms;

Z is a covalent bond, sulfonamido (—SO₂NR—), or carboxamido (—CONR—) where R is hydrogen or alkyl;

R¹ is an alkylene, heteroalkylene, aralkylene, or heteroaralkylene group;

R² is a divalent straight or branched chain alkylene, cycloalkylene, or heteroalkylene group of 1 to 14 carbon atoms, preferably 1 to 8 carbon atoms, more preferably 1 to 4 carbon atoms, and most preferably two carbon atoms, and preferably R² is alkylene or heteroalkylene of 1 to 14 carbon atoms;

Q is a multi-valent organic group that is a residue of the polyfunctional isocyanate compound;

R³ is a polyvalent, preferably divalent organic group which is a residue of the hydrophilic polyoxyalkylene;

X′ is —O—, —S—, or —N(R)—, wherein R is hydrogen or C₁-C₄ alkyl;

each Y is independently a hydroxy; a hydrolyzable moiety selected from the group consisting of alkoxy, acyloxy, heteroalkoxy, heteroacyloxy, halo, and oxime; or a non-hydrolyzable moiety selected from the group consisting of phenyl, alicyclic, straight-chain aliphatic, and branched-chain aliphatic, wherein at least one Y is a hydrolyzable moiety.

A is selected from the group consisting of R_{f}ZR²—OCONH—, (Y)₃SiR¹XCONH—, and (Y)₃SiR¹NHCOOR³OCONH—.

m is an integer from 0 to 2; and

n is an integer from 1 to 10.

It will be understood with respect to the above formulas that the compounds represent theoretical structures for the reaction products. The reaction product will contain a mixture of compounds in which the substitution patterns of the isocyanate groups will vary.

Polyfunctional isocyanate compounds useful in the present invention comprise isocyanate groups attached to the multivalent organic group, Q, which can comprise a multivalent aliphatic, alicyclic, or aromatic moiety; or a multivalent aliphatic, alicyclic or aromatic moiety attached to a blocked isocyanate, a biuret, an isocyanurate, or a uretdione, or mixtures thereof. Preferred polyfunctional isocyanate compounds contain at least two and preferably three or more —NCO groups. Compounds containing two —NCO groups are comprised of divalent aliphatic, alicyclic, araliphatic, or aromatic moieties to which the —NCO radicals are attached. Preferred compounds containing three —NCO radicals are comprised of isocyanatoaliphatic, isocyanatoalicyclic, or isocyanatoaromatic, monovalent moieties, which are attached to a biuret or an isocyanurate.

Representative examples of suitable polyfunctional isocyanate compounds include isocyanate functional derivatives of the polyfunctional isocyanate compounds as defined herein. Examples of derivatives include, but are not limited to, those selected from the group consisting of ureas, biurets, allophanates, dimers and trimers (such as uretdiones and isocyanurates) of isocyanate compounds, and mixtures thereof. Any suitable organic polyisocyanate, such as an aliphatic, alicyclic, araliphatic, or aromatic polyisocyanate, may be used either singly or in mixtures of two or more.

The aliphatic polyfunctional isocyanate compounds generally provide better light stability than the aromatic compounds, and are preferred for treatment of fibrous substrates. Aromatic polyfunctional isocyanate compounds, on the other hand, are generally more economical and reactive toward hydrophilic polyoxyalkylene compounds and other isocyanate-reactive compounds than are aliphatic polyfunctional isocyanate compounds.

Suitable aromatic polyfunctional isocyanate compounds include, but are not limited to, those selected from the group consisting of 2,4-toluene diisocyanate (TDI), 2,6-toluene diisocyanate, an adduct of TDI with trimethylolpropane (available as Desmodur™ CB from Bayer Corporation, Pittsburgh, Pa.), the isocyanurate trimer of TDI (available as Desmodur™ IL from Bayer Corporation, Pittsburgh, Pa.), diphenylmethane 4,4′-diisocyanate (MDI), diphenylmethane 2,4′-diisocyanate, 1,5-diisocyanato-naphthalene, 1,4-phenylene diisocyanate, 1,3-phenylene diisocyanate, 1-methyoxy-2,4-phenylene diisocyanate, 1-chlorophenyl-2,4-diisocyanate, and mixtures thereof.

Examples of useful alicyclic polyfunctional isocyanate compounds include, but are not limited to, those selected from the group consisting of dicyclohexylmethane disocyanate (H₁₂MDI, commercially available as Desmodur™W, available from Bayer Corporation, Pittsburgh, Pa.), 4,4′-isopropyl-bis(cyclohexylisocyanate), isophorone diisocyanate (IPDI), cyclobutane-1,3-diisocyanate, cyclohexane 1,3-diisocyanate, cyclohexane 1,4-diisocyanate (CHDI), 1,4-cyclohexanebis(methylene isocyanate) (BDI), 1,3-bis(isocyanatomethyl)cyclohexane (H₆XDI), 3-isocyanatomethyl-3,5,5-trimethylcyclohexyl isocyanate, and mixtures thereof.

Examples of useful aliphatic polyfunctional isocyanate compounds include, but are not limited to, those selected from the group consisting of 1,4-tetramethylene diisocyanate, hexamethylene 1,4-diisocyanate, hexamethylene 1,6-diisocyanate (HDI), 1,12-dodecane diisocyanate, 2,2,4-trimethyl-hexamethylene diisocyanate (TMDI), 2,4,4-trimethyl-hexamethylene diisocyanate (TMDI), 2-methyl-1,5-pentamethylene diisocyanate, dimer diisocyanate, the urea of hexamethylene diisocyanate, the biuret of hexamethylene 1,6-diisocyanate (HDI) (available as Desmodur™ N-100 and N-3200 from Bayer Corporation, Pittsburgh, Pa.), the isocyanurate of HDI (available as Demodur™ N-3300 and Desmodur™ N-3600 from Bayer Corporation, Pittsburgh, Pa.), a blend of the isocyanurate of HDI and the uretdione of HDI (available as Desmodurm N-3400 available from Bayer Corporation, Pittsburgh, Pa.), and mixtures thereof.

Examples of useful aryl aliphatic polyisocyanates include, but are not limited to, those selected from the group consisting of m-tetramethyl xylylene diisocyanate (m-TMXDI), p-tetramethyl xylylene diisocyanate (p-TMXDI), 1,4-xylylene diisocyanate (XDI), 1,3-xylylene diisocyanate, p-(1-isocyanatoethyl)-phenyl isocyanate, m-(3-isocyanatobutyl)-phenyl isocyanate, 4-(2-isocyanatocyclohexyl-methyl)-phenyl isocyanate, and mixtures thereof.

Preferred polyisocyanates, in general, include those selected from the group consisting of hexamethylene 1,6-diisocyanate (HDI), 1,12-dodecane diisocyanate isophorone diisocyanate, toluene diisocyanate, dicyclohexylmethane 4,4′-diisocyanate, MDI, derivatives of all the aforementioned, including Desmodur™ N-100, N-3200, N-3300, N-3400, N-3600, and mixtures thereof.

Suitable commercially available polyfunctional isocyanates are exemplified by Desmodur™ N-3200, Desmodur™ N-3300, Desmodur™ N-3400, Desmodur™ N-3600, Desmodur™ H (HDI), Desmodur™ W (bis[4-isocyanatocyclohexyl]methane), Mondur™ M (4,4′-diisocyanatodiphenylmethane), Mondur™ TDS (98% toluene 2,4-diisocyanate), Mondur™ TD-80 (a mixture of 80% 2,4 and 20% 2,6-toluene diisocyanate isomers), and Desmodur™ N−100, each available from Bayer Corporation, Pittsburgh, Pa.

Other useful triisocyanates are those obtained by reacting three moles of a diisocyanate with one mole of a triol. For example, toluene diisocyanate, 3-isocyanatomethyl-3,4,4-trimethylcyclohexyl isocyanate, or m-tetramethylxylene diisocyanate can be reacted with 1,1,1-tris(hydroxymethyl)propane to form triisocyanates. The product from the reaction with m-tetramethylxylene diisocyanate is commercially available as CYTHANE 3160 (American Cyanamid, Stamford, Conn.).

Fluorochemical monofunctional compounds suitable for use in preparing the chemical compositions of the present invention include those that comprise at least one R_{f} group. The R_{f} groups can contain straight chain, branched chain, or cyclic fluorinated alkylene groups or any combination thereof. The R_{f} groups can optionally contain one or more heteroatoms (i.e. oxygen, sulfur, and/or nitrogen) in the carbon-carbon chain so as to form a carbon-heteroatom-carbon chain (i.e. a heteroalkylene group). Fully-fluorinated groups are generally preferred, but hydrogen or chlorine atoms can also be present as substituents, provided that no more than one atom of either is present for every two carbon atoms. It is additionally preferred that any R_{f} group contain at least about 40% fluorine by weight, more preferably at least about 50% fluorine by weight. The terminal portion of the group is generally fully-fluorinated, preferably containing at least three fluorine atoms, e.g., CF₃O—, CF₃CF₂—, CF₃CF₂CF₂—, (CF₃)₂N—, (CF₃)₂CF—, SF₅CF₂—. In certain embodiments, perfluoroalkyl groups (i.e., those of the formula CₙF₂ₙ₊₁—) wherein n is 2 to 12 inclusive are the preferred R_{f} groups, with n=3 to 5 being more preferred and with n=4 being the most preferred.

Useful fluorochemical monofunctional compounds include compounds of the following formula:

R_{f}-Z-R²—X

wherein:

R_{f} is a perfluoroalkyl group or a perfluoroheteroalkyl group;

Z is a connecting group selected from a covalent bond, a sulfonamido group, a carboxamido group, a carboxyl group, or a sulfonyl group; and

R² is a divalent straight or branched chain alkylene, cycloalkylene, or heteroalkylene group of 1 to 14 carbon atoms, preferably 1 to 8 carbon atoms, more preferably 1 to 4 carbon atoms, and most preferably two carbon atoms, and

X is an isocyanate-reactive functional groups, for example —NH₂; —SH; —OH; —N═C═O; or —NRH where R is H or a C₁-C₄ alkyl.

Representative examples of useful fluorochemical monofunctional compounds include the following:

| | |
|---|---|
| CF₃(CF₂)₃SO₂N(CH₃)CH₂CH₂OH, | CF₃(CF₂)₃SO₂N(CH₃)CH(CH₃)CH₂OH, |
| CF₃(CF₂)₃SO₂N(CH₃)CH₂CH(CH₃)NH₂, | CF₃(CF₂)₃SO₂N(CH₂CH₃)CH₂CH₂SH, |
| CF₃(CF₂)₃SO₂N(CH₃)CH₂CH₂SCH₂CH₂OH, | C₆F₁₃SO₂N(CH₃)(CH₂)₄OH, |
| CF₃(CF₂)₇SO₂N(H)(CH₂)₃OH, | C₃F₇SO₂N(CH₃)CH₂CH₂OH, |
| CF₃(CF₂)₄SO₂N(CH₃)(CH₂)₄NH₂, | C₄F₉SO₂N(CH₃)(CH₂)₁₁OH, |
| CF₃(CF₂)₅SO₂N(CH₂CH₃)CH₂CH₂OH, | CF₃(CF₂)₅SO₂N(C₂H₅)(CH₂)₆OH, |
| CF₃(CF₂)₂SO₂N(C₂H₅)(CH₂)₄OH, | CF₃(CF₂)₃SO₂N(C₃H₇)CH₂OCH₂CH₂CH₂OH, |
| CF₃(CF₂)₄SO₂N(CH₂CH₂CH₃)CH₂CH₂OH, | CF₃(CF₂)₄SO₂N(CH₂CH₂CH₃)CH₂CH₂NHCH₃, |
| CF₃(CF₂)₃SO₂N(C₄H₉)CH₂CH₂NH₂, | CF₃(CF₂)₃SO₂N(C₄H₉)(CH₂)₄SH, |
| CF₃(CF₂)₃CH₂CH₂OH | C₄F₉OC₂F₄OCF₂CH₂OCH₂CH₂OH; |
| n-C₆F₁₃CF(CF₃)CON(H)CH₂CH₂OH; | C₆F₁₃CF(CF₃)CO₂C₂H₄CH(CH₃)OH; |
| C₃F₇CON(H)CH₂CH₂OH; | C₃F₇O(CF(CF₃)CF₂O)₁₋₃₆CF(CF₃)CH₂OH; |

and the like, and mixtures thereof. If desired, other isocyanate-reactive functional groups may be used in place of those depicted.

Certain preferred embodiments of the chemical compositions of the present invention include those compositions comprising terminal fluoroalkyl groups having from two to twelve carbons, preferably from three to six carbons, and more preferably four carbons. Even with relatively short perfluoroalkyl groups (i.e. six or fewer carbons), these chemical compositions, surprisingly, exhibit excellent release/resistance/repellency. Although compositions comprising lower fluorine content are less expensive, those of skill in the art have typically overlooked R_{f} groups shorter than eight carbons because they have been known to impart inferior oil- and water-repellency and stain resistance.

Many previously known fluorochemical surfactants contain perfluorooctyl moieties. These surfactants ultimately degrade to perfluorooctyl-containing compounds. It has been reported that certain perfluorooctyl-containing compounds may tend to bio-accumulate in living organisms; this tendency has been cited as a potential concern regarding some fluorochemical compounds. For example, see U.S. Pat. No. 5,688,884 (Baker et al.). As a result, there is a desire for fluorine-containing compositions which are effective in providing desired release/resistance/repellency properties, and which eliminate more effectively from the body (including the tendency of the composition and its degradation products).

It is expected that the preferred fluorochemical compositions of the present invention, which contain perfluoroalkyl C₃ to C₆ moieties, when exposed to biologic, thermal, oxidative, hydrolytic, and photolytic conditions found in the environment, will break down to various degradation products. For example, compositions comprising perfluorobutylsulfonamido moieties are expected to degrade, at least to some extent, ultimately to perfluorobutylsulfonate salts. It has been surprisingly found that perfluorobutylsulfonate, tested in the form of its potassium salt, eliminates from the body much more effectively than perfluorohexylsulfonate and even more effectively than perfluorooctylsulfonate.

Useful perfluoroheteroalkyl groups correspond to the formula:

R¹_{f}—O—R_{f}²—(R_{f}³)_{q}— (II)

wherein R¹_{f} represents a perfluorinated alkyl group, R_{f}² represents a perfluorinated polyalkyleneoxy group consisting of perfluorinated alkyleneoxy groups having 1, 2, 3 or 4 carbon atoms or a mixture of such perfluorinated alkylene oxy groups, R³_{f} represents a perfluorinated alkylene group and q is 0 or 1. The perfluorinated alkyl group R¹_{f} in formula (II) may be linear or branched and may comprise 1 to 10 carbon atoms, preferably 1 to 6 carbon atoms. A typical perfluorinated alkyl group is CF₃—CF₂—CF₂—. R³_{f} is a linear or branched perfluorinated alkylene group that will typically have 1 to 6 carbon atoms. For example, R³_{f} is —CF₂— or —CF(CF₃)—. Examples of perfluoroalkylene oxy groups of perfluorinated polyalkyleneoxy group R²_{f} include:

—CF₂—CF₂—O—,

—CF(CF₃)—CF₂—O—,

—CF₂—CF(CF₃)—O—,

—CF₂—CF₂—CF₂—O—,

—CF₂—O—,

—CF(CF₃)—O—,

—CF₂—CF₂—CF₂—CF₂—O.

The perfluoroalkyleneoxy group may be comprised of the same perfluoroalkylene oxy units or of a mixture of different perfluoroalkylene oxy units. When the perfluoroalkyleneoxy group is composed of different perfluoroalkylene oxy units, they can be present in a random configuration, alternating configuration or they can be present as blocks. Typical examples of perfluorinated poly(alkyleneoxy) groups include: —[CF₂—CF₂—O]ᵣ—; —[CF(CF₃)—CF₂—O]ₙ—; —[CF₂CF₂—O]ᵢ—[CF₂O]ⱼ— and —[CF₂—CF₂—O]ₗ—[CF(CF₃)—CF₂—O]ₘ—; wherein r is an integer of 4 to 25, n is an integer of 3 to 25 and i, l, m and j each are integers of 2 to 25. A preferred perfluorinated polyether group that corresponds to formula (II) is CF₃—CF₂—CF₂—O—[CF(CF₃)—CF₂O]ₙ—CF(CF₃)— wherein n is an integer of 3 to 25. This perfluorinated polyether group has a molecular weight of 783 when n equals 3 and can be derived from an oligomerization of hexafluoropropylene oxide. Such perfluorinated polyether groups are preferred in particular because of their benign environmental properties.

Examples of the moiety “-Z-R²-” include organic groups that comprise aromatic or aliphatic groups that may be interrupted by O, N or S and that may be substituted, alkylene groups, oxy groups, thio groups, urethane groups, carboxy groups, carbonyl groups, amido groups, oxyalkylene groups, thioalkylene groups, carboxyalkylene and/or an amidoalkylene groups. Examples of functional groups T include thiol, hydroxy and amino groups.

In a particular embodiment, the fluorochemical monofunctional compound corresponds to the following formula:

R_{f}¹—[CF(CF₃)—CF₂O]ₙ—CF(CF₃)-Z-R²—X

wherein R_{f}¹ represents a perfluorinated alkyl group e.g. a linear or branched perfluorinated alkyl group having 1 to 6 carbon atoms, n is an integer of 3 to 25, Z is a carbonyl group or CH₂, R² is a chemical bond or an organic divalent or trivalent linking group for example as mentioned for the linking group Q above, and X represents an isocyanate reactive group and each X may be the same or different. Particularly preferred compounds are those in which R¹_{f} represents CF₃CF₂CF₂—. In accordance with a particular embodiment, the moiety -Z-R²—X is a moiety of the formula —CO—X—R^{a}(OH)ₖ wherein k is 1 or 2, X is O or NR^{b} with R^{b} representing hydrogen or an alkyl group of 1 to 4 carbon atoms, and R^{a} is an alkylene of 1 to 15 carbon atoms.

Representative examples of the moiety “-Z-R²—X” in above formula (III) include: —CONR—CH₂CHOHCH₂OH wherein R is hydrogen or an alkyl group of, for example, 1 to 4 carbon atoms; —CONH-1,4-dihydroxyphenyl; —CH₂OCH₂CHOHCH₂OH; —COOCH₂CHOHCH₂OH and —CONR—(CH₂)ₘOH

where R² is hydrogen or an alkyl group of, for example, 1 to 4 carbon atoms.

Perfluoropolyether monofunctional compounds can be obtained by oligomerization of hexafluoropropylene oxide that results in a perfluoropolyether carbonyl fluoride. This carbonyl fluoride may be converted into an acid, ester or alcohol by reactions well known to those skilled in the art. The carbonyl fluoride or acid, ester or alcohol derived therefrom may then be reacted further to introduce the desired isocyanate reactive groups according to known procedures. For example, EP 870 778 describes suitable methods to produce compounds having desired moieties “-Z-R²—. Compounds having group 1 listed above can be obtained by reacting the methyl ester derivative of a fluorinated polyether with 3-amino-2-hydroxy-propanol. Compounds having the group 5 listed above can be obtained in a similar way by reacting with an amino-alcohol that has only one hydroxy function. For example 2-aminoethanol would yield a compound having the group 5 listed above with R² being hydrogen and m being 2. Still further examples of compounds according to above formula (I) are disclosed in EP 870 778 or U.S. Pat. No. 3,536,710.

It will be evident to one skilled in the art that a mixture of fluorinated polyethers according to formula (I) may be used to prepare the fluorinated polyether compound of the fluorochemical composition. Generally, the method of making the perfluoropolyether according to formula (I) will result in a mixture of perfluoropolyether that have different molecular weights and such a mixture can be used as such to prepare the fluorochemical component of the fluorochemical composition. In a preferred embodiment, such a mixture of perfluoropolyether compounds is free of fluorinated polyether compounds having a perfluorinated polyether moiety having a molecular weight of less than 750 g/mol or alternatively the mixture contains fluorinated polyether compounds having a perfluorinated polyether moiety having a molecular weight of less than 750 g/mol in an amount of not more than 10% by weight relative to total weight of fluorinated polyether compounds, preferably not more than 5% by weight and most preferably not more than 1% by weight.

Hydrophilic polyoxyalkylene compounds, if present, suitable for use in preparing the first component fluorochemical urethane compounds of the present invention include those polyoxyalkylene compounds that have an average functionality of greater than 1 (preferably, about 2 to 5; more preferably, about 2 to 3; most preferably, about 2, as difunctional compounds such as diols are most preferred). The isocyanate-reactive groups can be primary or secondary, with primary groups being preferred for their greater reactivity. Mixtures of compounds having different functionalities, for examples mixtures of polyoxyalkylene compounds having one, two and three isocyanate-reactive groups, may be used provide the average is greater than 1. The polyoxyalkylene groups include those having 1 to 3 carbon atoms such as polyoxyethylene, polyoxypropylene, and copolymers thereof such as polymers having both oxyethylene and oxypropylene units.

Examples of polyoxyalkylene containing compounds include alkyl ethers of polyglycols such as e.g. methyl or ethyl ether of polyethylene glycol, hydroxy terminated methyl or ethyl ether of a random or block copolymer of ethylene oxide and propylene oxide, amino terminated methyl or ethyl ether of polyethyleneoxide, polyethylene glycol, polypropylene glycol, a hydroxy terminated copolymer (including a block copolymer) of ethylene oxide and propylene oxide, a mono- or diamino-terminated poly(alkylene oxide) such as Jeffamine™ ED, Jeffamine™ EDR-148 and poly(oxyalkylene) thiols. Commercially available aliphatic polyisocyanates include Baygard™ VP SP 23012, Rucoguard™ EPF 1421 and Tubicoat™ Fix ICB.

Useful commercially available hydrophilic polyoxyalkylene compounds for the first component include Carbowax™ poly(ethylene glycol) materials in the number average molecular weight (Mₙ) range of from about 200 to about 2000 (available from Dow Chemical Corp.); poly(propylene glycol) materials such as PPG-425 (available from Lyondell Chemicals); block copolymers of poly(ethylene glycol) and poly(propylene glycol) such as Pluronic™ L31 (available from BASF Corporation); the “PeP” series (available from Wyandotte Chemicals Corporation) of polyoxyalkylene tetrols having secondary hydroxyl groups, for example, “PeP” 450, 550, and 650.

Silane compounds, if present, suitable for use in the chemical compositions of the present invention are those of the following formula:

X—R¹—Si—(Y)₃

wherein X is an isocyanate-reactive functional group such as —NH₂; —SH; —OH; —N═C═O;

or —NRH where R is H or a C₁-C₄ alkyl;

R¹ is an alkylene, heteroalkylene, aralkylene, or heteroaralkylene group; and

each Y is independently a hydroxy; a hydrolyzable moiety selected from the group consisting of alkoxy, acyloxy, heteroalkoxy, heteroacyloxy, halo, and oxime; or a non-hydrolyzable moiety selected from the group consisting of phenyl, alicyclic, straight-chain aliphatic, and branched-chain aliphatic, wherein at least one Y is a hydrolyzable moiety.

Therefore, these silane compounds contain one, two, or three hydrolysable groups (Y) on the silicon and one organic group including an isocyanate-reactive or an active hydrogen reactive radical (X—R¹). Any of the conventional hydrolysable groups, such as those selected from the group consisting of alkoxy, acyloxy, heteroalkoxy, heteroacyloxy, halo, oxime, and the like, can be used as the hydrolyzable group (Y). The hydrolysable group (Y) is preferably alkoxy or acyloxy and more preferably alkoxy.

When Y is halo, the hydrogen halide liberated from the halogen-containing silane can cause polymer degradation when cellulose substrates are used. When Y is an oxime group, lower oxime groups of the formula —N═CR⁵R⁶, wherein R⁵ and R⁶ are monovalent lower alkyl groups comprising about 1 to about 12 carbon atoms, which can be the same or different, preferably selected from the group consisting of methyl, ethyl, propyl, and butyl, are preferred.

Representative divalent bridging radicals (R¹) include, but are not limited to, those selected from the group consisting of —CH₂CH₂—, —CH₂CH₂CH₂—, —CH₂CH₂CH₂OCH₂CH₂—, —CH₂CH₂C₆H₄CH₂CH₂—, and —CH₂CH₂O(C₂H₄O)₂CH₂CH₂N(CH₃)CH₂CH₂CH₂—.

Other preferred silane compounds are those which contain one or two hydrolysable groups, such as those having the structures R²OSi(R⁷)₂R¹XH and (R⁸O)₂Si(R⁷)R¹XH, wherein R¹ is as previously defined, and R⁷ and R⁸ are selected from the group consisting of a phenyl group, an alicylic group, or a straight or branched aliphatic group having from about 1 to about 12 carbon atoms. Preferably, R⁷ and R⁸ are a lower alkyl group comprising 1 to 4 carbon atoms.

Following the hydrolysis of some of these terminal silyl groups, inter-reaction with a substrate surface comprising —SiOH groups or other metal hydroxide groups to form siloxane linkages, e.g.,

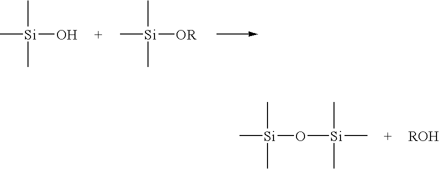

can occur. Bonds thus formed, particularly Si—O—Si bonds, are water resistant and can provide enhanced durability of the stain-release properties imparted by the chemical compositions of the present invention.

Such silane compounds are well known in the art and many are commercially available or are readily prepared. Representative isocyanate-reactive silane compounds include, but are not limited to, those selected from the group consisting of:

| | |
|---|---|
| H₂NCH₂CH₂CH₂Si(OC₂H₅)₃; | H₂NCH₂CH₂CH₂Si(OCH₃)₃; |
| H₂NCH₂CH₂CH₂Si(O—N═C(CH₃)(C₂H₅))₃ | HSCH₂CH₂CH₂Si(OCH₃)₃; |
| HO(C₂H₄O)₃C₂H₄N(CH₃)(CH₂)₃Si(OC₄H₉)₃; | H₂NCH₂C₆H₄CH₂CH₂Si(OCH₃)₃; |
| HSCH₂CH₂CH₂Si(OCOCH₃)₃; | HN(CH₃)CH₂CH₂Si(OCH₃)₃; |
| HSCH₂CH₂CH₂SiCH₃(OCH₃)₂; | (H₃CO)₃SiCH₂CH₂CH₂NHCH₂CH₂CH₂Si(OCH₃)₃; |
| HN(CH₃)C₃H₆Si(OCH₃)₃; | CH₃CH₂OOCCH₂CH(COOCH₂CH₃)HNC₃H₆Si(OCH₂CH₃)₃; |
| C₆H₅NHC₃H₆Si(OCH₃)₃; | H₂NC₃H₆SiCH₃(OCH₂CH₃)₂; |
| HOCH(CH₃)CH₂OCONHC₃H₆Si(OCH₂CH₃)₃; | (HOCH₂CH₂)₂NCH₂CH₂CH₂Si(OCH₂CH₃)₃ |
| H₂NCH₂CH₂NHCH₂CH₂CH₂Si(OC₂H₅)₃ | H₂NCH₂CH₂NHCH₂CH₂CH₂Si(OCH₃)₃ |

and mixtures thereof.

The treatment compositions of this invention contain silsesquioxanes. Useful silsesquioxanes include co-condensates of diorganooxysilanes (or hydrosylates thereof) of the formula R¹⁰₂Si(OR¹¹)₂ with organosilanes (or hydrosylates thereof) of the formula R¹⁰SiO_{3/2} where each R¹⁰ is an alkyl group of 1 to 6 carbon atoms or an aryl group and R¹¹ represents an alkyl radical with 1 to 4 carbon atoms. Preferred silsesquioxanes are neutral or anionic silsesquioxanes, prior to addition to the composition. Useful silsesquioxanes can be made by the techniques described in U.S. Pat. No. 3,493,424 (Mohrlok et al.), U.S. Pat. No. 4,351,736 (Steinberger et al.), U.S. Pat. No. 5,073,442 (Knowlton et al.) U.S. Pat. No. 4,781,844 (Kortmann, et al), and U.S. Pat. No. 4,781,844, each incorporated herein by reference.

The silsesquioxanes may be prepared by adding silanes to a mixture of water, a buffer, a surface active agent and optionally an organic solvent, while agitating the mixture under acidic or basic conditions. It is preferable to add the quantity of silane uniformly and slowly in order to achieve a narrow particle size of 200 to 500 Angstroms. The exact amount of silane that can be added depends on the substituent R and whether an anionic or cationic surface-active agent is used. Co-condensates of the silsesquioxanes in which the units can be present in block or random distribution are formed by the simultaneous hydrolysis of the silanes. The amount of tetraorganosilanes, including tetralkoxysilan-es and hydrosylates thereof (e.g. of the formula Si(OH)₄) present is less than 10 wt. %, preferably less than 5 wt. %, more preferably less than 2 wt. % relative to the weight of the silsesquioxane.

The following silanes are useful in preparing the silsesquioxanes of the present invention: methyltrimethoxysilane, methyltriethoxysilane, methyltriisopropoxyoxysilane, ethyltrimethoxysilane, ethyltriethoxysilane, propyltrimethoxysilane, isobutyltrimethoxysilane, isobutyltriethoxysilane, 2-ethylbutyltriethoxysilane, and 2-ethylbutoxytriethoxysilane.

Generally, the hydroxy number is from about 1000 to 6000 per gram, and is preferably from about 1500 to 2500. The hydroxy number may be measured, for example, by titration or the molecular weight may be estimated by ²⁹Si NMR.

The composition may contain no more than 10 wt. % of Si(OH)₄ or tetraorganooxysilanes or hydrosylates therof, (preferably less than 5 wt. %, more preferably less than 2 wt. %) which the inventors have found to degrade the oil repellency performance of the resulting fluorochemical composition. Most commercial siloxane resins contain amounts in excess of this amount due to the incorporation of tetralkyoxysilanes in the cocondensate. A particularly useful silsesquioxane containing essentially no residual tetralkyoxysilanes (or hydrosylates thereof such as Si(OH)₄) is SR 2400 Resin™ available from Dow Corning, Midland, Mich.

The fluorochemical compositions of the present invention may be made according to the following step-wise synthesis. As one skilled in the art would understand, the order of the steps is non-limiting and can be modified so as to produce a desired chemical composition. In the synthesis, the polyfunctional isocyanate compound and the monofunctional fluorochemical compound are dissolved together under dry conditions, preferably in a solvent, and then heating the resulting solution at approximately 40 to 80° C., preferably approximately 60 to 70° C., with mixing in the presence of a catalyst for one-half to two hours, preferably one hour. Depending on reaction conditions (e.g., reaction temperature and/or polyfunctional isocyanate used), a catalyst level of up to about 0.5 percent by weight of the polyfunctional isocyanate/monofunctional fluorochemical compound mixture may be used, but typically about 0.00005 to about 0.5 percent by weight is required, 0.02 to 0.1 percent by weight being preferred.

Suitable catalysts include, but are not limited to, tertiary amine and tin compounds. Examples of useful tin compounds include tin II and tin IV salts such as stannous octanoate, dibutyltin dilaurate, dibutyltin diacetate, dibutyltin di-2-ethylhexanoate, and dibutyltinoxide. Examples of useful tertiary amine compounds include triethylamine, tributylamine, triethylenediamine, tripropylamine, bis(dimethylaminoethyl) ether, morpholine compounds such as ethyl morpholine, and 2,2′-dimorpholinodiethyl ether, 1,4-diazabicyclo[2.2.2]octane (DABCO, Aldrich Chemical Co., Milwaukee, Wis.), and 1,8-diazabicyclo[5.4.0.]undec-7-ene (DBU, Aldrich Chemical Co., Milwaukee, Wis.). Tin compounds are preferred.

The resulting fluorochemical functional urethane compounds are optionally further reacted with one or more of the silane compounds described above. The silane compound is added to the above reaction mixture, and reacts with a substantial portion of the remaining NCO groups. The above temperatures, dry conditions, and mixing are continued one-half to two hours, preferably one hour. Terminal silane-containing groups are thereby bonded to the isocyanate functional urethane compounds. Aminosilanes are preferred, because of the rapid and complete reaction that occurs between the remaining NCO groups and the silane compound's amino groups. Isocyanato functional silane compounds may be used and are preferred when the ratio of polyfunctional isocyanate compound to the hydrophilic difunctional polyoxyalkylene and fluorochemical monofunctional compound is such that the resulting compound has a terminal hydroxyl group.

These compounds are optionally further functionalized with polyoxyalkylene compounds, having an average functionality of greater than 1, described above by reacting any of the remaining NCO groups in the resulting mixture with one or more of the reactive polyoxyalkylene compounds described above. Thus, the polyoxyalkylene compound(s) is (are) added to the reaction mixture, using the same conditions as with the previous additions.

Optionally, any unreacted isocyanate groups may be hydrolysed, esterified or may comprise a blocked isocyanate group. A “blocked isocyanate” is a polyisocyanate of a portion of the isocyanate groups have been reacted with a blocking agent. Isocyanate blocking agents are compounds that upon reaction with an isocyanate group yield a group that is unreactive at room temperature with compounds that at room temperature normally react with an isocyanate but which group at elevated temperature reacts with isocyanate reactive compounds. Generally, at elevated temperature the blocking group will be released from the blocked polyisocyanate group thereby generating the isocyanate group again which can then react with an isocyanate reactive group, such as may be found on the surface of a fibrous substrate. Blocking agents and their mechanisms have been described in detail in “Blocked isocyanates III.: Part. A, Mechanisms and chemistry” by Douglas Wicks and Zeno W. Wicks Jr., Progress in Organic Coatings, 36 (1999), pp. 14-172.

The blocked isocyanate may be aromatic, aliphatic, cyclic or acyclic and is generally a blocked di- or triisocyanate or a mixture thereof and can be obtained by reacting an isocyanate with a blocking agent that has at least one functional group capable of reacting with an isocyanate group. Preferred blocked isocyanates are blocked polyisocyanates that, at a temperature of less than 150° C., are capable of reacting with an isocyanate reactive group, through deblocking of the blocking agent at elevated temperature. Preferred blocking agents include arylalcohols such as phenols, lactams such as ε-caprolactam, δ-valerolactam, γ-butyrolactam, oximes such as formaldoxime, acetaldoxime, methyl ethyl ketone oxime, cyclohexanone oxime, acetophenone oxime, benzophenone oxime, 2-butanone oxime or diethyl glyoxime. Further suitable blocking agents include bisulfite and triazoles.

Alternatively, any unreacted isocyanate group remaining may be hydrolyzed to an amine (or salt thereof), or may be reacted with an alcohol to form a urethane group.

For aqueous systems the fluorochemical urethane treatment composition of this invention may further comprise polymers of acrylic and/or methacrylic monomers, which are found to impart durability to the treatment. Particular examples of such polymers include homo- and copolymers of alkyl esters of acrylic and methacrylic acid such as for example C₁ to C₃₀ alkyl esters of acrylic acid. Specific examples of such alkyl esters include methyl acrylate, ethyl acrylate, butyl acrylate, octadecyl acrylate and lauryl acrylate. Specific examples of suitable polymers include a homopolymer of methyl acrylate and a copolymer of methyl acrylate and octadecyl acrylate. Commercially available acrylic copolymers include the Hybridur™ products available from Air Products (Allentown, Pa.). Such acrylate copolymers may generally be used in amounts of 2 to 20% relative to the weight % of the fluorochemical urethane.

The composition may further comprise a crosslinking catalyst for the silsesquioxane component. Examples of suitable crosslinking agent include organotitanates, organogermanates and organozirconates. The crosslinking catalyst have the general structure M(OR¹²)₄, where M is a titanium, germanium or zirconium atom and each R¹² is a monovalent hydrocarbon radical or acyl radical. The R¹² radical can be any alkyl, aryl, alkenyl, aralkyl, aminoalkyl, oxaalkyl, hydroxyalkyl, and alkaryl radical as well as acyl. Each R¹² can be the same or different and mixtures of catalysts may be used. The catalyst may be used in amounts of 0.01 to 3 wt. %, preferably 0.1 to 2 wt. %, relative to the weight of the silsesquioxane.

Useful catalysts include alkyl tin derivatives, e.g., dibutyltindilaurate, dibutyltindiacetate, and dibutyltindioctoate commercially available as “T-series Catalysts” from Air Products and Chemicals, Inc. of Allentown, Pa.), ortho zirconate esters (e.g., n-butyl zirconate, n-propyl zirconate) and ortho titanate esters (e.g., tetraisobutylorthotitanate, titanium acetylacetonate, and acetoacetic ester titanate commercially available from DuPont under the designation “TYZOR”). Such crosslinking agents are particularly useful for composition containing organic solvents, but titanate compounds for aqueous systems are also known (e.g., titanium lactate chelate, TYZOR LA). Reference may be made to U.S. Pat. No. 2,732,320 (Guillissen et al.), U.S. Pat. No. 3,647,846 (Hartlein et al.), U.S. Pat. No. 3,015,637 (Rauner et al.) and U.S. Pat. No. 4,399,247 (Ona et al.), each incorporated herein by reference.

The coating composition for fibrous substrates comprises a solution or dispersion of the chemical compositions of the present invention and at least one solvent. When applied to fibrous substrates, the treatment compositions impart release/resistance/repellency characteristics and exhibit durability (i.e. they resist being worn-off) when exposed to wear and abrasion from use, cleaning, and the elements.

The fluorochemical compositions of the present invention can be dissolved or dispersed in a variety of solvents to form coating compositions suitable for use in coating the chemical compositions of the present invention onto a substrate. Fibrous substrate treatment compositions may contain from about 0.1 to about 50 weight percent chemical composition. Preferably the chemical composition is used in the coating composition at about 0.1 to about 10 weight percent, most preferably from about 2 to about 4 weight percent.

Suitable solvents include water, alcohols, esters, glycol ethers, amides, ketones, hydrocarbons, chlorohydrocarbons, chlorocarbons, and mixtures thereof. Depending upon the substrate to which the composition is being applied, water is the preferred solvent because it does not raise any environmental concerns and is accepted as safe and non-toxic. Mixtures of solvents may be used, however such mixtures should be selected so that the fluorochemical urethane and the silsesquioxane components are soluble in the least volatile solvent, to provide uniform coating of the composition.

The treatment compositions of the present invention can be applied as to a wide variety of fibrous substrates resulting in an article that displays durable stain-release properties. The article of the present invention comprises a fibrous substrate having a treatment derived from at least one solvent and a chemical composition of the present invention. After application and curing of the coating composition, the substrate displays durable release/repellency/resistance properties.

The treatment composition may also be applied to other substrates including glass, ceramic, stone, metal, semi-porous materials such as grout, cement and concrete, wood, paint, plastics, rubber.

The treatment compositions can be applied to a wide variety of fibrous substrates including woven, knit, and nonwoven fabrics, textiles, carpets, leather, and paper. Substrates having nucleophilic groups, such as cotton are preferred because they can bond to the silane groups and/or isocyanate groups of the chemical compositions of the present invention, thereby increasing durability of the fiber treatment. Any application method known to one skilled in the art can be used including spraying, dipping, immersion, foaming, atomizing, aerosolizing, misting, flood-coating, and the like.

To impart release/repellency/resistance characteristics to a fibrous substrate, the coating composition of the present invention is applied to the substrate and is allowed to cure (i.e. dry), at ambient or elevated temperature.

The treating process for applying the composition can be either an exhaustion process or a topical process. In the exhaustion process, a fibrous substrate is first treated exhaustively by contacting the entirety of each fiber of the substrate with the aqueous composition of this invention. Following the contacting step, the resulting totally wet fibrous substrate is then heated in a water-saturated atmosphere such as a steam box for a time sufficient to affix the treating composition onto each fiber surface. The heated wet fibrous substrate is subsequently rinsed with water and is dried in an oven at sufficient temperature to effectively activate the treating composition on the surface of each fiber. In some cases, application at a sufficient high bath temperature (e.g., over 200° F. (83° C.)) can eliminate the need for the post-steaming operation. The fibrous substrate, having had total penetration throughout each fiber, exhibits significant protection against soiling when compared to untreated carpet as demonstrated by several cycles of “walk-on” tests, and exhibits excellent dynamic water resistance (i.e., the treated carpet resists penetration by water-based drinks spilled from a height).

Examples of suitable exhaustion processes for treating fibrous substrates include immersion, flooding, Beck vat processing, hot otting, padding and puddle foaming application. Useful treating equipment includes equipment available from Eduard Kusters Maschinefabrik GmbH & Co. KG, Krefeld, Germany, such as Kuster's Flex-nip™ equipment, Kuster's foam applicator, Fluicon™ flood applicator and Fluidye™ unit.

Suitable topical treating processes for applying the aqueous acidic composition comprising silsesquioxane include spraying and low density foam application. For example, the chemical composition can be applied by spraying the composition on the fibrous substrate. An ambient cure preferably takes place at approximately 15 to 35° C. (i.e. ambient temperature) until dryness is achieved, up to approximately 24 hours. With either heat-treatment or ambient cure, the chemical composition can also form chemical bonds with the substrate and between molecules of the chemical composition. However, exhaustion treating processes are preferred as they impart superior performance to the treated fibers.

If desired, the treated fibrous substrate maybe subjected to a heat treatment, for example, in an oven. A heat treatment is typically carried out at temperatures between about 50° C. and about 190° C. depending on the particular system or application method used. In general, a temperature of about 120° C. to 170° C., in particular of about 150° C. to about 170° C. for a period of about 20 seconds to 10 minutes, preferably 3 to 5 minutes, is suitable. With either heat-treatment or ambient cure, the chemical composition can also form chemical bonds with the substrate and between molecules of the chemical composition.

The choice of either heat-treatment or ambient cure often depends on the desired end-use. For consumer applications, where the composition may be applied to household laundry, upholstery or carpeting, an ambient cure is desired. For industrial applications, where the fibrous substrate, such as a textile might normally be exposed to elevated temperatures during production, an elevated temperature cure or heat-treatment may be desirable. Generally, those composition containing blocked isocyanate groups are preferred where a heat-treatment is encountered

The amount of the treating composition applied to the fibrous substrate is chosen so that a sufficiently high level of the desired properties are imparted to the substrate surface without substantially affecting the look and feel of the treated substrate. Such amount is usually such that the resulting amount of the fluorochemical urethane composition on the treated fibrous substrate will be between 0.05% and 10%, preferably 0.1 to 5% by weight based on the weight of the fibrous substrate, known as solids on fiber or SOF. The amount that is sufficient to impart desired properties can be determined empirically and can be increased as necessary or desired.

Suitable fibrous substrates include carpet, fabric, textiles and any substrate woven from fibers such as yarn or thread; carpet is the preferred form of the fibrous substrate. The fiber can be made from any number of thermoset or thermoplastic polymers, such as polyamide, polyester, acrylic and polyolefin; cellulose. Polyamide (e.g. nylon) is the preferred fiber.

The resulting treated substrates derived from at least one solvent and a chemical composition of the present invention, have been found to be resist soils and/or stains and/or to release soils and/or stains with simple washing methods. The cured treatments have also been found to be durable and hence to resist being worn-off due to wear and abrasion from use, cleaning, and the elements.

The invention will now be further illustrated with reference to the following examples without the intention to limit the invention thereto. All parts and percentages are by weight unless stated otherwise.

### EXAMPLES

TABLE 1

| Designation | Material | Availability/Preparation |
|---|---|---|
| APTES | 3-aminopropyltriethoxysilane; | Sigma-Aldrich, Milwaukee, WI |
| | NH₂(CH₂)₃Si(OC₂H₅)₃ | |
| Ethyl acetate | CH₃CO₂C₂H₅ | Sigma-Aldrich |
| DBTDL | Dibutyltin dilaurate; | Sigma-Aldrich |
| | [CH₃(CH₂)₃]₂Sn[CO₂(CH₂)₁₀CH₃]₂ | |
| MeFBSE | N-methylperfluorobutanesulfonyl | Prepared by reacting |
| | ethanol; C₄F₉SO₂N(CH₃)CH₂CH₂OH | perfluorobutanesulfonyl fluoride |
| | | with CH₃NH₂ and ethylene |
| | | chlorohydrin, essentially as |
| | | described in Ex. 1 of U.S. Pat. No. |
| | | 2,803,656 (Ahlbrecht, et al.) |
| MIBK | Methylisobutyl ketone; | Sigma-Aldrich |
| | (CH₃)₂CHCH₂C(O)CH₃ | |
| DESMODUR ™ | Polyfunctional isocyanate resin based | Bayer, Pittsburgh, PA |
| N-3300 | on hexamethylene diisocyanate | |
| | eq wt = 194 | |
| CARBOWAX ™ | Polyethylene glycol (MWₐᵥ = 1450) | Dow Chemical Company, |
| PEG 1450 | | Midland, MI |
| SR-2400 ™ | Methyl silsesquioxane hydroxy | Dow Corning Corporation, |
| | terminated dimethylsiloxane polymer | Midland, MI |
| | OH # = 2000/g | |
| Dow Corning | Polydimethylsiloxane polymer | Dow Corning Corporation, |
| 200 ® Fluid | 350 centistokes (cst) | Midland, MI |
| Dow Corning ® 2- | Silicone resin | Dow Corning Corporation, |
| 7466 | | Midland, MI |
| Tyzor ® TOT | 2-Ethylhexyl titanate | E. I. DuPont de Nemours and |
| | | Company, Wilmington, DE |
| Rhodacal ® DS-10 | sodium dodecylbenzene sulfonate | Rhodia Inc., Marietta, GA |
| Isopar ™ E | Isoparaffinic fluid | Exxon Mobil Chemical Co., |
| | | Houston, TX, |
| Methyl acetate | CH₃CO₂CH₃ | Sigma-Aldrich, Milwaukee, WI |
| Acetone | CH₃COCH₃ | Sigma-Aldrich, Milwaukee, WI |
| Isopropyl alcohol | CH₃CHOHCH₃ | Sigma-Aldrich, Milwaukee, WI |
| (IPA) | | |
| Lambent Amine | Water dispersible amino silicone | Lambent Technologies, |
| PD | softener | Fernandina Beach, FL |
| Hybridur ® 580 | Acrylic-urethane hybrid polymer | Air Products, Allentown, PA |
| | dispersion | |

Fabrics

Fabrics tested included: 100% cotton fabric (#428 from Test Fabrics Inc. Middlesex, N.J.) and a 100% polyolefin velveteen fabric (A8, Cherokee Finishing Co., NJ).

Application & Testing of Compositions

Performance Test—Oil Repellency

This test measures the resistance of the treated fabric to oil-based challenges. A drop of one standard surface tension fluid (of a series of 8, with decreasing surface tensions) is dropped on a treated fabric. If after thirty seconds there is no wetting, the next highest standard number fluid (next lowest surface tension) is tested. When the lowest number fluid soaks into the fabric, the next lower number is the rating. For example, the fabric will receive a three rating, if the number four fluid wets the fabric. A more detailed description of the test is written in the 3M Protective Material Division's “Oil Repellency Test I” method (Document # 98-0212-0719-0).

Performance Test—Water Repellency:

This test measures the resistance of the treated fibrous substrates to water based challenges. A drop of one standard surface tension fluid (of a series of 11, with decreasing surface tensions, based on water and water/isopropyl alcohol mixtures where 100% water is a 0 rating and 100% EPA is a 10 rating) is placed on a treated fabric to form a bead. If after thirty seconds there is no wetting, the next highest standard number fluid (next lowest surface tension) is tested. When the lowest number fluid soaks into the fabric, the next lower number is the rating. For example, the fabric will receive a three rating, if the number four fluid wets the fabric. A more detailed description of the test is written in the 3M Protective Material Division's “Water Repellency Test II” method (Document # 98-0212-0721-6).

Performance Test—Repellency Durability Test

The purpose of this test is to measure oil or water repellency after abrasion using a Crock Meter fitted with an abrasive disk. A more detailed description of the test is written in the 3M Home Care Division's “Spray and Oil Repellency Rating” method (Document # HHP-TM-48).

Performance Test—Spray Test

The purpose of this test is to measure what is called the dynamic spray rating. The test procedure is modified from AATCC Test Method 42-1980. The test is conducted by dropping room temperature water (250 ml) on a test fabric at a 45 degree incline. A rating of 100 is given if the all of the water runs off of the sample and rating of 50 is given if there is significant wetting of the surface of the fabric. A more detailed description of the test is written in the 3M Home Care Division's “Spray and Oil Repellency Rating” method (Document # HHP-TM-40).

Performance Test—Wetness Test

The purpose of this test is to measure the wet through of water after conducting the Spray test. The rating scale is 1 to 6 where a rating of 6 is completely dry and a rating of 1 is completely wet. A more detailed description of the test is written in the 3M Home Care Division's “Spray and Oil Repellency Rating” method (Document # HHP-TM-40).

Performance Test—Artificial Antisoiling

This test measures the resistance of the treated fibrous substrates to soil challenges. Typically a 12 in by 18 in sample of carpet is divided into three to six sections. One section is left untreated as the control and the other are treated with a protective finish and let dry at room temperature where T is = or <100 degrees F. and relative humidity is <50%. The treated article is taped to the inside of a drum filled with 40 ceramic pellets half weighing 10 g and half weighing 20 g and 20 g of 3M standard oily test soil (available from 3M Protective Materials Division Product 41-4201-6292-1). The drum is rolled for 10 minutes and then rolled the other opposite direction for 10 minutes. The carpet is removed and vacuumed in two directions and the treated areas are compared with an untreated area. Direct comparisons are made within the same sample and are rated from 1 to 5 where 1 is untreated (significant soiling) and 5 is no soiling. A more detailed description of the test is written in the AATCC “Artificial Antisoiling Test” method 123-1995.

Performance Test—Acid Stain Resistance

This test measures the resistance of the treated fibrous substrates to red acid dye stain. Test Method AATCC TM 175-1998 was followed. The rating given is compared to untreated virgin carpet where a rating of 1 is untreated and a rating of 5 indicates complete stain removal.

Preparation of Fluorochemical (FC) Urethane

FC Urethane MeFBSE/N3300/PEG 1450/APTES

The following preparation procedure was typical. A 1 liter flask was charged with of MeFBSE (58.89 g), DBTDL (3 drops; ˜20 mg) and ethyl acetate (237.0 g check amounts for all). The temperature of the stirred mixture was raised to 60° C. under a purge of dry nitrogen. N3300 (40.0 g) was then slowly added, maintaining the temperature between 60-65 C. Upon completion of the addition, the reaction mixture was stirred for 1 hour at 60° C. APTES (4.56 g) was then added dropwise, keeping temperature of the reaction mixture below 65° C., and the reaction mixture was stirred for 30 minutes. Solid PEG 1450 (12.00 g) was added to the stirred mixture, and the reaction was followed to completion via FTIR, as determined by disappearance of the —NCO band at approximately 2289 cm⁻¹. Any unreacted —NCO groups were then quenched with methanol or ethanol.

Testing on Fabrics

The FC urethane was combined with the SR-2400 silsesquioxane to give the compositions listed in Table 2. These were typically done at room temperature in a vented hood. The typical order of addition was the FC urethane, then the diluent, the silsesquioxane, and then the crosslinker. If filled in a pressurized can, then CO₂ is added last, however, it is not necessary to deliver from a pressurized can for performance. All percentages in the Table are weight percent solids unless otherwise indicated.

TABLE 2

| Preparation | SR- | FC | Tyzor ® | Lambent | | Methyl | | Iospropyl | |
|---|---|---|---|---|---|---|---|---|---|
| No. | 2400 | Urethane | TOT | Amine PD | Isopar ™ E | acetate | Acetone | alcohol | CO₂ |
| 1 | 0.98 | 2.5 | 0.25 | — | 22.00 | 38.00 | — | 30.00 | 4.00 |
| 2 | 0.98 | 2.5 | 0.50 | — | 31.57 | 38.00 | — | 20.00 | 4.00 |
| 3 | 0.70 | 1.8 | 0.50 | — | 31.57 | 38.00 | — | 20.00 | 4.00 |
| 4 | 0.84 | 2.2 | 0.50 | — | 31.57 | 38.00 | — | 20.00 | 4.00 |
| 5 | 0.98 | 2.5 | 0.50 | 0.50 | 31.57 | — | 37.50 | 20.00 | 4.00 |
| 6 | 0.98 | 2.5 | 0.50 | 1.00 | 31.57 | — | 37.00 | 20.00 | 4.00 |
| 7 | 0.98 | 2.5 | 0.50 | 1.50 | 31.57 | — | 36.50 | 20.00 | 4.00 |
| 8 | 0.98 | 2.5 | 0.45 | 1.00 | 31.10 | — | 38.00 | 20.00 | 4.00 |
| 9 | 0.83 | 2.1 | 0.38 | 0.85 | 32.13 | — | 38.00 | 20.00 | 4.00 |
| 10 | 0.69 | 1.8 | 0.32 | 0.70 | 33.17 | — | 38.00 | 20.00 | 4.00 |
| C1 | 0.98* | 2.5 | 0.50 | — | 31.57 | 38.00 | — | 20.00 | 4.00 |
| C2 | 0.98** | 2.5 | 0.50 | — | 31.57 | 38.00 | — | 20.00 | 4.00 |

*For C1, Dow Corning 200 ® fluid 350 cst was used in place of SR-2400
**For C2, Dow Corning ® 2-7466 was used in place of SR-2400

Preparations 1-10 were applied to the #428 Cotton test fabric as an aerosolized spray to yield a 20 g/ft² wet add-on. Test results for static water repellency (Performance Test—Water Repellency), static oil repellency (Performance Test—Oil Repellency), wetness (Performance Test—Wetness), spray (Performance Test—Spray Test) and durability (Performance Test—Repellency Durablity) are listed in Table 3 for Examples 1-10 and Comparative Examples C1 and C2.

TABLE 3

| | | Static | | | | Durability | Durability |
|---|---|---|---|---|---|---|---|
| | Preparation | Water | Static Oil | | | (Water | (Oil |
| Ex. | No. | Repellency | Repellency | Wetness | Spray | Repellency) | Repellency) |
| 1 | 1 | 6 | 7 | 6 | 65 | 6 | 6 |
| 2 | 2 | 6 | 7 | 6 | 75 | N/C | N/C |
| 3 | 3 | 4 | 5 | 6 | 60 | 4 | 4 |
| 4 | 4 | 5 | 5 | 6 | 60 | 5 | 4 |
| 5 | 5 | 6 | 6 | 6 | 65 | — | — |
| 6 | 6 | 6 | 5 | 6 | 65 | 6 | 5 |
| 7 | 7 | 5 | 4 | 6 | 60 | — | — |
| 8 | 8 | 5 | 5 | 6 | 65 | — | — |
| 9 | 9 | 5 | 5 | 6 | 65 | — | — |
| 10 | 10 | 4 | 5 | 5 | 65 | — | — |
| Control | (untreated) | 0 | 0 | 1 | 0 | — | — |
| C1 | C1 | 1 | 0 | 5 | 60 | — | — |
| C2 | C2 | 3 | 4 | 6 | 60 | — | — |

Preparations 1-10 were applied to the A8 polyolefin velveteen test fabric as an aerosolized spray to yield a 20 g/ft² wet add-on. Test results for static water repellency (Performance Test—Water Repellency), static oil repellency (Performance Test—Oil Repellency), wetness (Performance Test—Wetness), spray (Performance Test—Spray), and durability (Performance Test—Repellency Durability Test) are listed in Table 4 for Examples 11-20.

TABLE 4

| | | Static | | | | Durability | Durability |
|---|---|---|---|---|---|---|---|
| | | Water | Static Oil | | | (Water | (Oil |
| Ex. | Prep. No. | Repellency | Repellency | Wetness | Spray | Repellency) | Repellency) |
| 11 | 1 | 5 | 5 | 6 | 65 | 5 | 5 |
| 12 | 2 | 7 | 6 | 6 | 70 | 6 | 6 |
| 13 | 3 | 5 | 5 | 6 | 60 | 4 | 5 |
| 14 | 4 | 6 | 6 | 6 | 65 | 5 | 6 |
| 15 | 5 | 6 | 6 | 6 | 70 | 5 | 6 |
| 16 | 6 | 6 | 6 | 6 | 70 | 5 | 6 |
| 17 | 7 | 6 | 6 | 6 | 70 | 5 | 6 |
| 18 | 8 | 6 | 6 | 6 | 70 | 6 | 6 |
| 19 | 9 | 6 | 6 | 6 | 70 | 5 | 6 |
| 20 | 10 | 5 | 6 | 6 | 65 | 4 | 5 |

Testing on Carpet

The fluorochemical urethane was combined with the SR-2400 silsesquioxane to give Preparations 11-15 listed in Table 5 as solvent borne (IPA) systems at the % solids indicated (approximately 25% solids). Preparations 11-15 were subsequently diluted to 3-4$ solids emulsions with deionized water to yield ready-to-use compositions, which were then applied to a carpet as a spray (Blue Transition III Nylon 6,6 virgin carpet available from Shaw Industries, Dalton, Ga.) to yield an approximately 0.4-0.7 g/ft² solids add-on. Test results for static water repellency (Performance Test—Water Repellency), static oil repellency (Performance Test—Oil Repellency), anti-soiling (Performance Test—Anti-soiling), and anti-staining (Performance Test—Acid Stain Resistance) are listed in Table 6 for Examples 21-25. All percentages in the Table are weight percent solids unless otherwise indicated.

TABLE 5

| | % | | | FC | | |
|---|---|---|---|---|---|---|
| | Solids | | | Urethane: | Water | % Solids |
| Prep. | (in | SR- | FC | SR- | dilution | (Ready-to- |
| No. | IPA) | 2400 | Urethane | 2400 ratio | ratio | use) |
| 11 | 27 | 6.75 | 20.0 | 3:1 | 1:6 | 3.9 |
| 12 | 27 | 6.75 | 20.0 | 3:1 | 1:7 | 3.4 |
| 13 | 27 | 6.75 | 20.0 | 3:1 | 1:8 | 3.0 |
| 14 | 22.6 | 4.71 | 17.9 | 3.8:1 | 1:6 | 3.8 |
| 15 | 22.6 | 4.71 | 17.9 | 3.8:1 | 1:8 | 3.2 |

TABLE 6

| | | Static | | | |
|---|---|---|---|---|---|
| | Preparation | Water | Static Oil | Anti- | Acid Stain |
| Ex. | No. | Repellency | Repellency | soiling | Resistance |
| 21 | 11 | 1 | 3.5 | 8 | 3 |
| 22 | 12 | 1 | 3.5 | 7.5 | 3 |
| 23 | 13 | 1 | 2.5 | 6 | 3 |
| 24 | 14 | 0.5 | 1 | 6 | 3 |
| 25 | 15 | 0.5 | 1 | 6.5 | 3 |
| Control | (untreated) | 0 | 0 | 1 | 1 |

The water-diluted systems described above were not stable in water and also showed lack of durability to vacuuming. Preparations 16-18 listed in Table 7 were prepared as solvent free emulsions containing an added acrylate (Hybridurm 580) for improved durability to vacuuming. Comparative Preparation C3 contained no SR-2400 silsesquioxane and no added acrylate. In some instances (Preparation 17 and Comparative Preparation C3) silicone fluid was added for additional stability. The emulsions were prepared as follows:

To a 1 gallon jar were added 588 g of the fluorochemical urethane (65% in ethyl acetate) and 52 g of the silsesquioxane (65% in methylisobutyl ketone). This mixture was heated to 150° F. In a separate jar, 1000 g of water was warmed to 160° F. and in this was dissolved 65 g of Rhodacal™ DS-10. Upon dissolution and reaching appropriate temperatures the water was combined with the fluorochemical and silsesquioxane solutions under high speed stirring. The waterborne acrylate emulsion was then added and further stirred. The mixture was then homogenized and stripped en vacuo at 160° F. bath temperature. Upon complete removal of the solvents the resulting solids contents was approximately 30 to 40%. The mixtures were then further diluted with deionized water to give ready-to-use compositions at approximately 3% solids. An optional silicone fluid or other solvent may be added prior to homogenization or after stripping. As indicated in Table 7, Preparation 17 and Comparative Preparation C3 included silicone fluid for additional stability.

Preparations 16-18 and Comparative Preparation C3 were subsequently diluted to 3-4% solids emulsions with deionized water to yield ready-to-use compositions, which were then applied to a carpet as a spray (Blue Transition III Nylon 6,6 virgin carpet available from Shaw Industries, Dalton, Ga.) to yield an approximately 0.4-0.7 g/ft² solids add-on. Test results for static water repellency (Performance Test—Water Repellency), static oil repellency (Performance Test—Oil Repellency), anti-soiling (Performance Test—Anti-soiling), and anti-staining (Performance Test—Acid Stain Resistance) are listed in Table 8 for Examples 26-28 and Comparative Example C3. All percentages in the Table are weight percent solids unless otherwise indicated.

TABLE 7

| | | | | | | | % |
|---|---|---|---|---|---|---|---|
| | | | | | | Water | (Solids |
| Preparation | FC | SR- | Hybridur ® | Rhodocal | Silicone | dilution | Ready- |
| no. | Urethane | 2400 | 580 | DS-10 | fluid* | ratio | to-use) |
| 16 | 82.6 | 7.3 | 7.3 | 2.8 | 0 | 1:8 | 3.1 |
| 17 | 76.5 | 6.8 | 6.8 | 2.8 | 10% (D4) | 1:8 | 3.2 |
| 18 | 76.5 | 6.8 | 6.8 | 2.8 | 0 | 1:8 | 2.9 |
| C3 | 97.5 | 0 | 0 | 2.5 | 10% (D5) | 1:8 | 3.0 |

*Cyclic —(CH₃)₂SiO)x— = D4 when X = 4, = D5 when X = 5

TABLE 8

| | | | Water | | Oil | | |
|---|---|---|---|---|---|---|---|
| | Preparation | Water | repellency | Oil | repellency | Anti- | Anti- |
| Ex. | No. | repellency | (time) | repellency | (time) | staining | soiling |
| 26 | 16 | 1 | >20 min | 1 | 16 min | 3 | 3 |
| 27 | 17 | 2 | >20 min | 1 | 10 min | 3 | 3 |
| 28 | 18 | 0 | 14 min | Fail | 0 | 3 | 2 |
| C3 | C3 | 1 | 4 min | Fail | 0 | 2 | 4 |
| Control | untreated | 0 | — | 0 | — | 1 | 1 |

What is claimed is:

1. A composition comprising:
(a) a first component comprising one or more fluorochemical urethane compounds comprising the reaction product of:
(1) one or more polyfunctional isocyanate compounds; and
(2) one or more fluorochemical monofunctional compounds; and
(b) a second component comprising one or more silsesquioxanes, wherein said silsesquioxane comprises less than 10 wt. % of cocondensates of tetraalkoxysilanes or hydrosylates thereof.

2. The composition of claim 1, wherein said silsesquioxanes comprise co-condensates of silanes (or hydrosylates thereof) of the formula R¹⁰₂Si(OR¹¹)₂ with silanes of the formula R¹⁰SiO_{3/2} where each R¹⁰ is an alkyl group of 1 to 6 carbon atoms or an aryl group and each R¹¹ represents an alkyl radical with 1 to 4 carbon atoms.

3. The composition of claim 2 wherein said silsesquioxane has a hydroxy number of 1000 to 6000.

4. The composition of claim 1 wherein said first component further comprises the reaction product of one or more hydrophilic polyoxyalkylene compounds.

5. The composition of claim 1 wherein said first component further comprises the reaction product of one or more silane compounds of the formula:
X—R¹—Si—(Y)₃
wherein
X is —SH; —OH; —N═C═O; or —NRH where R is selected from the group consisting of phenyl, straight and branched aliphatic, alicyclic, and aliphatic ester groups;
R¹ is an alkylene, heteroalkylene, aralkylene, or heteroaralkylene group; and
each Y is independently a hydroxyl; a hydrolyzable moiety selected from the group consisting of alkoxy, acyloxy, heteroalkyoxy, heteroacyloxy, halo, and oxime; or a non-hydrolyzable moiety selected from the group consisting of phenyl, alicyclic, straight-chain aliphatic, and branched-chain aliphatic, wherein at least one Y is a hydrolyzable moiety.

6. The chemical composition of claim 1 wherein the polyfunctional isocyanate compound of said first component is a diisocyanate, triisocyanate or mixture thereof.

7. The chemical composition of claim 1 wherein the fluorochemical monofunctional compound of said first component is of the formula:
R_{f}-Z-R²—X
wherein:
R_{f} is a perfluoroalkyl group or a perfluoroheteroalkyl group;
Z is a connecting group selected from a covalent bond, a sulfonamido group, a carboxamido group, a carboxyl group, or a sulfonyl group; and
R² is a divalent straight or branched chain alkylene, cycloalkylene, or heteroalkylene group of 1 to 14 carbon atoms; and
X is —NH₂; —SH; —OH; —N═C═O; or —NRH where R is selected from the group consisting of phenyl, straight and branched aliphatic, alicyclic, and aliphatic ester groups; R¹ is an alkylene, heteroalkylene, aralkylene, or heteroaralkylene group.

8. The chemical composition of claim 7 wherein R_{f} is a perfluoroalkyl group of 2 to 12 carbons.

9. The chemical composition of claim 7 wherein R_{f} is a perfluoroalkyl group of 3 to 5 carbons.

10. The composition of claim 7 wherein R_{f} is a perfluoropolyether.

11. The composition of claim 4 wherein said first component polyoxyalkylene compounds are homo- and copolymers of polyoxyethylene and polyoxypropylene.

12. The composition of claim 4 herein the amount of said hydrophilic polyoxyalkylene compounds of said first component is sufficient to react with between 0.1 and 30% of available isocyanate groups, the amount of said silane compounds is sufficient to react with between 0.1 and 25% of available isocyanate groups, and the amount of said fluorochemical monofunctional compounds is sufficient to react with between 60 and 95% of available isocyanate groups of said urethane compounds.

13. The composition of claim 1 wherein any unreacted isocyanate groups are blocked isocyanate groups.

14. The composition of claim 1 wherein the ratio of said first component to said second component is from 1:1 to 10:1.

15. The composition of claim 1 wherein the weight ratio of said first component to said second component is from 3:1 to 5:1.

16. The composition of claim 1 comprising 50 to 90 wt. % of said first component and 10 to 50 wt. % of said second component.

17. A treatment composition comprising a solution of the chemical composition of claim 1 and a solvent.

18. The treatment composition of claim 17 wherein the solvent is selected from the group consisting of water, an organic solvent, and mixtures thereof.

19. The treatment composition of claim 17 comprising from 0.1 to about 50 weight percent composition comprising a mixture of said fluorochemical urethane and said silsequioxane.

20. The treatment composition of claim 17 comprising 50 to 90 wt. % of said first component and 10 to 50 wt. % of said second component.

21. An article comprising a fibrous substrate having a cured coating derived from at least one solvent and a chemical composition of claim 1.

22. The article of claim 21 wherein said fibrous substrate comprises natural and synthetic fibrous substrates.

23. The article of claim 22 wherein said synthetic fibrous substrate comprises polyamide, polyester, acrylic and polyolefin fibers.

24. A method for imparting stain-release characteristics to a fibrous substrate comprising the steps of applying the treatment composition of claim 17, and allowing the coating composition to cure.

25. The method of claim 24 wherein said coating composition is applied in an amount sufficient to provide between 0.05% and 5% solids on fiber.

26. The method of claim 24 wherein said composition is cured at ambient temperature.
